(19) United States
(12) Patent Application Publication
(10) Pub. No.: US 2006/0189932 A1
(43) Pub. Date: Aug. 24, 2006

(54) Safety syringe

(76) Inventors: Jih-Hsiung Yang, Taichung Hsien (TW); Hsiu-Chih Lin, Taichung Hsien (TW)

Correspondence Address: DELLETT & WALTERS, P. O. BOX 82788, PORTLAND, OR, 97282-0788, US

(21) Appl. No.: 11/052,973

(22) Filed: Feb. 7, 2005

(51) Int. Cl. A61M 5/00; A61M 5/32

(52) U.S. Cl. 604/110; 604/192

(57) ABSTRACT

The safety syringe has a hollow barrel, a needle hub, a sleeve and a plunger. The needle hub is mounted on the hollow barrel and has a needle, a hub and a stopper. The needle is mounted on the hub and the stopper is mounted inside the hub. The hub has channels through which medication or blood can pass. The sleeve is mounted on the needle hub. The plunger is mounted slidably inside the hollow barrel. The used needle is retracted into the tube after use by a vacuum after seating the stopper and retracting the plunger to keep people from getting injured or infected.

### BACKGROUND OF THE INVENTION

1. Field of the Invention

The present invention relates to a syringe, and more particularly to a safety syringe that can safely hold a used needle and prevent the syringe from being used more than once.

2. Description of Related Art

Due to contagious diseases, needles of syringes and even the hollow barrels and plungers, should not be used again and should be discarded immediately after use. Also, to keep nurses, doctors or medical workers who deal with discarded syringes from getting injured or infected by used needles, safety syringes are needed.

A conventional safety syringe has a hollow barrel, a plunger and a needle hub. To prevent a used needle from injuring a user, the needle hub is pulled back inside the hollow barrel. The needle hub of the conventional safety syringe often has some locking elements formed on the needle hub to lock the needle hub to the plunger so the used needle hub can be pulled back inside the hollow barrel. However, the structure of the locking elements formed on the needle hub is often complex. Further, the complex structure increases the cost and results in safety syringes not being available to all.

To overcome the shortcomings of conventional safety syringes, the present invention provides a safety syringe to mitigate or obviate the aforementioned problem.

### SUMMARY OF THE INVENTION

The primary objective of the present invention is to provide a safety syringe that has a simpler structure than the conventional safety syringe and improves safety.

The safety syringe in accordance with the present invention comprises a hollow barrel, a needle hub, a sleeve and a plunger. The needle hub is mounted on the hollow barrel and has a needle, a hub and a stopper. The needle is mounted on the hub and the stopper is mounted inside the hub. The hub has channels through which medication or blood can pass. The sleeve is mounted on the needle hub. The plunger is mounted slidably inside the hollow barrel. The used needle is retracted into the tube after use by a vacuum after seating the stopper and retracting the plunger to keep people from getting injured or infected.

Other objectives, advantages and novel features of the invention will become more apparent from the following detailed description when taken in conjunction with the accompanying drawings.

### BRIEF DESCRIPTION OF THE DRAWINGS

FIG. 1 is an exploded perspective view of a first embodiment of a safety syringe in accordance with the present invention;

FIG. 2 is a side view in partial section of the safety syringe in FIG. 1;

FIG. 3 is a cross sectional rear view of the safety syringe along line 3-3 in FIG. 2;

FIG. 4 is a side view in partial section of the safety syringe in FIG. 1 with the plunger totally pressed completely into the hollow barrel;

FIG. 5 is a side view in partial section of the safety syringe in FIG. 1 with the cap being loosened from the needle hub;

FIG. 6 is a side view in partial section of the safety syringe in FIG. 1 with the plunger being broken;

FIG. 7 is a side view in partial section of a second embodiment of a safety syringe in accordance with the present invention; and

FIG. 8 is a cross sectional rear view of the safety syringe along line 8-8 in FIG. 7.

### DETAILED DESCRIPTION OF THE PREFERRED EMBODIMENT

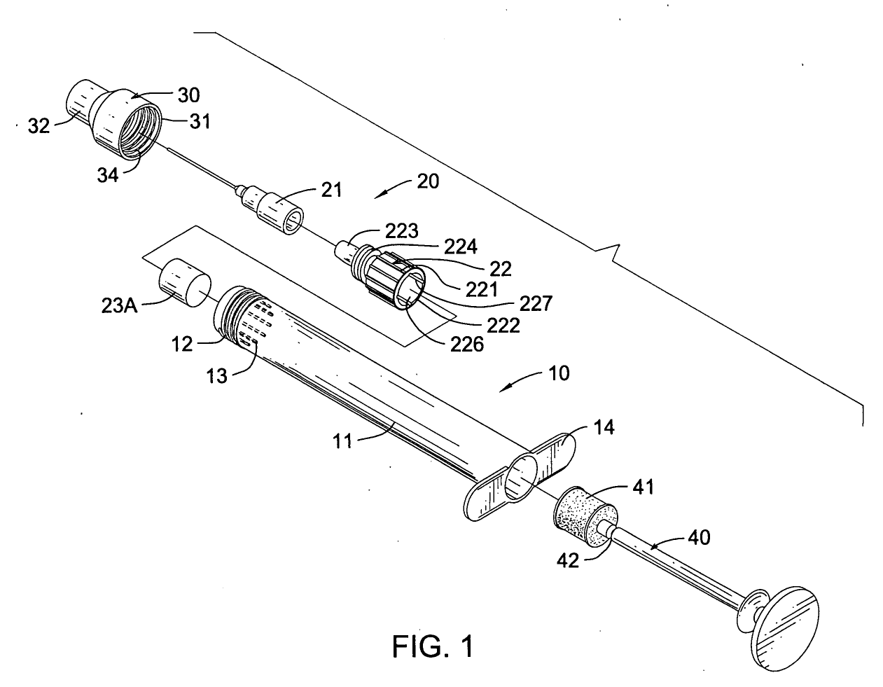

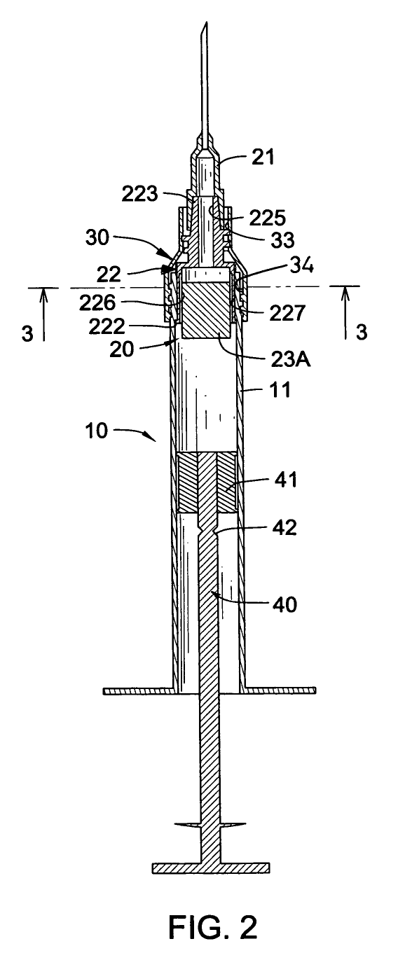

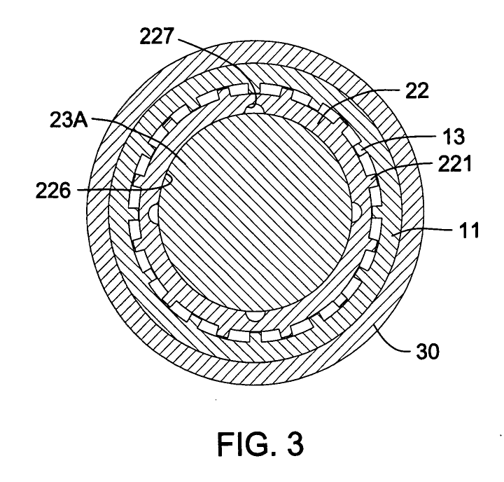

With reference to FIGS. 1 to 3, a first embodiment of a safety syringe (10) in accordance with the present invention has a hollow barrel (11), a needle hub (20), a sleeve (30) and a plunger (40).

The hollow barrel (11) is cylindrical and has a distal end, a proximal end, an outside surface, an inner surface, an exterior thread (12), multiple longitudinal ribs (13) and a finger flange (14). The exterior thread (12) is formed on the outside surface near the distal end. The longitudinal ribs (13) are formed on the inner surface near the distal end. The finger flange (14) is defined at the proximal end and protrudes from diametrically opposite sides.

The needle hub (20) is mounted on the hollow barrel (11) at the distal end and has a needle (21), a hub (22) and a stopper (23A). The needle (21) is mounted on the hub (22) and has a fluid channel defined though the needle (21). The hub (22) has a proximal end, a distal end (223), a middle, an outside surface, an inner surface, multiple longitudinal ribs (221), an optional flange (222), an external thread (224), a fluid channel (225), a cavity (226) and at least one channel (227). In a preferred embodiment, the distal end (223) is tapered and is mounted inside the needle (21). In a certain embodiment, the needle (21) may directly form on the distal end of the hub (22). The longitudinal ribs (221) are formed on the outside surface and engaged by the longitudinal ribs (13) inside the hollow barrel (11). The flange (222) is formed at the proximal end and extends out from the proximal end to seal the hollow barrel (11). The external thread (224) is formed at the middle of the hub (20) near the distal end (223). The fluid channel (225) is defined through the distal end (223) and the external thread (224). The cavity (226) is defined through the hub (22) and communicates with the fluid channel (225) and has a depth. The at least one channel (227) is defined in the inner surface of the hub (22), communicates with the cavity (226) and has a length. In a preferred embodiment, four channels (227) are defined in the inner surface of the hub (22). In a preferred embodiment, the length of the channel (227) is shorter than the depth of the cavity (226). The stopper (23A) is slidably mounted inside the cavity (226) in the hub (22) and has a height. In a preferred embodiment, the height of the stopper (23A) is equal to the depth of the cavity (226).

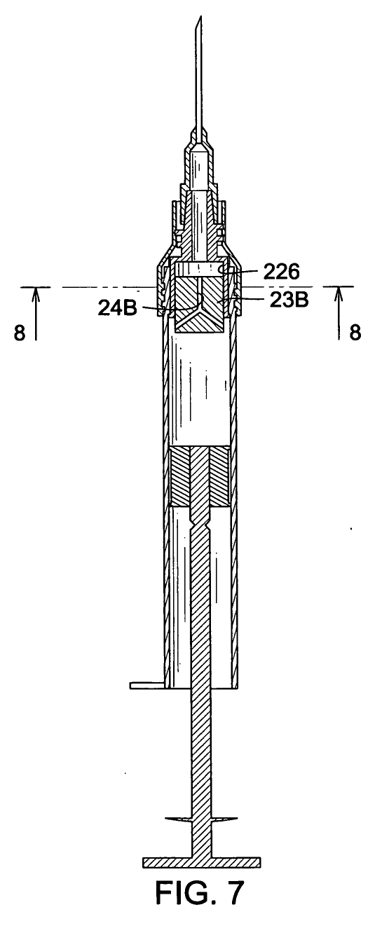

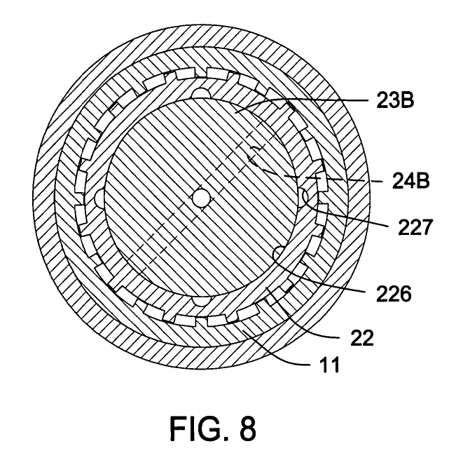

With further reference to FIGS. 7 and 8, a second embodiment of the safety syringe in accordance with the present invention has a stopper (23B) with a transverse Y-shaped channel (24B) for liquid medication or blood can pass through quickly. In a preferred embodiment, the height of the stopper (23B) is shorter than the depth of the cavity (226). Before using the safety syringe, the stopper (23A, 23B) is partially mounted inside the cavity (226).

The sleeve (30) is mounted on the hub (22) and has an inner surface, a front end (32), a rear end (31), an outer interior thread (33) and an inner interior thread (34). The outer interior thread (33) is defined in the inner surface at the front end (32), corresponds to and screws onto the external thread (224) on the hub (22). The inner interior thread (34) is defined in the inner surface at the rear end (31), corresponds to and screws onto the threaded end (12) of the hollow barrel (11).

The plunger (40) is slidably mounted inside the hollow barrel (11) and has a distal end, a proximal end, a sliding stopper (41) and an optional annular notch (42). The sliding stopper (41) is mounted on the distal end of the plunger (40), and the annular notch (42) is defined in the plunger (40) near the sliding stopper (41).

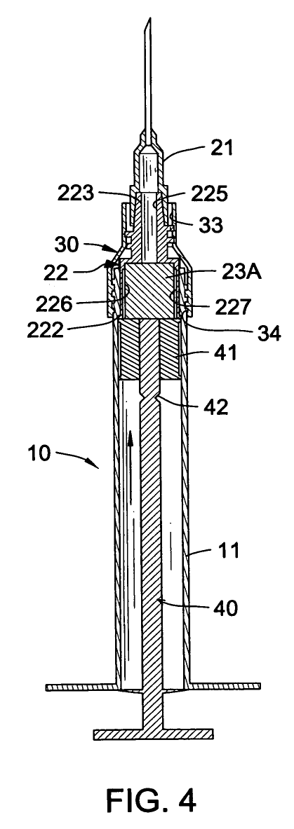

With further reference to FIG. 4, the safety syringe is filled with medication by pulling the plunger (40) back to draw the medication through the fluid channel (225) and the channels (227) into the hollow barrel (11). To inject the medication, the plunger (40) is pushed, and the medication moves alone the channels (227), the fluid channel (225) and the needle (21).

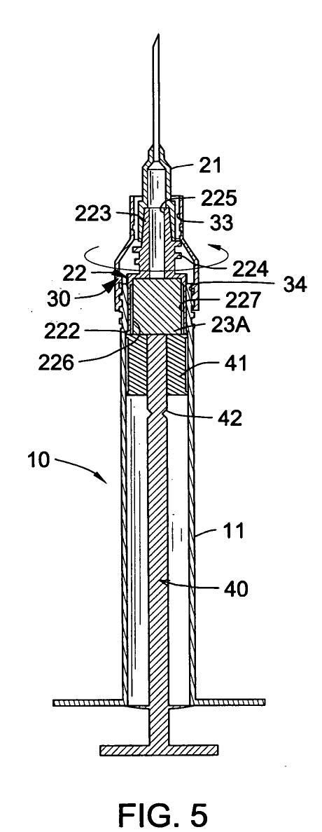

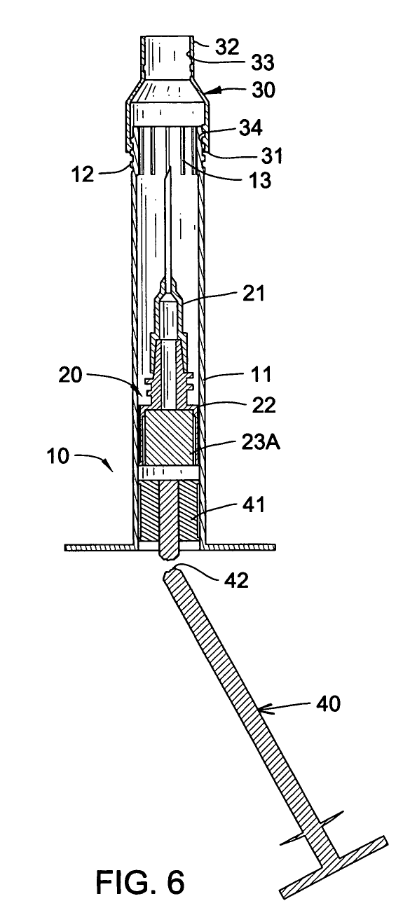

With reference to FIGS. 4 to 6, the safety features of the safety syringe are implemented by pushing the stopper (23A) firmly into the cavity (226), unscrewing the sleeve (30) and retracting the plunger (40). The stopper (23A) seals the channels (227) and the fluid channel (225) and keeps the fluid channel (225) from communicating with the cavity (226) in the hub (22). The cap (30) can be unscrewed from the needle hub (22) because the longitudinal ribs (221) on the hub (22) engage with the longitudinal ribs (13) on the inner surface of the hollow barrel (11) and keep the hub (22) from rotating. The plunger (40) is pulled back, which generates a vacuum in the hollow barrel (11) pulls the needle (21) into the hollow barrel (40). Then the plunger (40) is broken at the annular notch (42) to keep the needle (21) from being pushed out of the hollow barrel (40) again.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A safety syringe comprising;
a hollow barrel having
a distal end,
a proximal end,
an outside surface,
an inner surface,
an exterior thread formed on the outside surface near the distal end,
multiple longitudinal ribs formed on the inner surface near the distal end, and
a finger flange defined at the proximal end and protrudes from diametrically opposite sides,
a needle hub mounted on the hollow barrel at the distal end and having
a needle having a fluid channel defined though the needle,
a hub connected to the needle and having
a proximal end,
a distal end,
a middle,
an outside surface,
an inner surface,
multiple longitudinal ribs formed on the outside surface and engaged by the longitudinal ribs inside the hollow barrel,
an external thread formed at the middle of the hub near the distal end,
a fluid channel defined through the distal end and the external thread and communicating with the fluid channel in the needle,
a cavity defined through the hub, communicating with the fluid channel in the hub and having a depth, and
at least one channel defined in the inner surface of the hub, and communicating with the cavity and having a length, and
a stopper slidably mounted inside the cavity in the hub and having a height,
a sleeve mounted on the hub and having
an inner surface,
a front end,
a rear end,
an outer interior thread defined in the inner surface at the front end, corresponding to and screwing onto the external thread on the hub, and
an inner interior thread defined in the inner surface at the rear end, corresponding to and screwing onto the threaded end of the hollow barrel, and
a plunger slidably mounted inside the hollow barrel and having
a distal end,
a proximal end, and
a sliding stopper mounted on the distal end of the plunger.

2. The safety syringe as claimed in claim 1, wherein the distal end of the hub is tapered and mounted inside the needle.

3. The safety syringe as claimed in claim 1, wherein the hub further comprises a flange formed at the proximal end and extending out from the proximal end to seal the hollow barrel.

4. The safety syringe as claimed in claim 1, wherein the stopper has a transverse Y-shaped channel defined in the stopper.

5. The safety syringe as claimed in claim 1, wherein four channels are defined in the inner surface of the hub.

6. The safety syringe as claimed in claim 1, wherein the length of the channel is shorter than the depth of the cavity.

7. The safety syringe as claimed in claim 1, wherein the plunger further comprises an annular notch defined in the plunger near the sliding stopper.

8. The safety syringe as claimed in claim 2, wherein the hub further comprises a flange formed at the proximal end and extends out from the proximal end to seal up the hollow barrel.

9. The safety syringe as claimed in claim 8, wherein four channels are respectively defined in the inner surface of the hub.

10. The safety syringe as claimed in claim 9, wherein the plunger further comprises an annular notch defined in the plunger near the sliding stopper.
